(19) United States
(12) Patent Application Publication
(10) Pub. No.: US 2015/0005855 A1
(43) Pub. Date: Jan. 1, 2015

(54) LIQUID TITANIUM FABRIC ARTICLES AND METHODS OF USE THEREOF

(71) Applicant: Wilhelmina McEwan, Rembert, SC (US)

(72) Inventor: Wilhelmina McEwan, Rembert, SC (US)

(21) Appl. No.: 14/315,805

(22) Filed: Jun. 26, 2014

Related U.S. Application Data
(60) Provisional application No. 61/841,803, filed on Jul. 1, 2013.

(51) Int. Cl. A61N 5/00

(52) U.S. Cl. CPC A61N 5/00
USPC 607/100

(57) ABSTRACT

Systems, apparatus, and methods for medical and therapeutic applications using liquid titanium fabrics are described. Systems and methods may include a method of reducing symptoms of an ailment. The method may include providing an article including liquid titanium fabric, and applying the fabric to a patient for a period of time effective to reduce symptoms of an ailment.

### CROSS-REFERENCE TO RELATED APPLICATIONS

This application claims priority to U.S. Provisional Patent Application No. 61/841,803, filed Jul. 1, 2013, the content of which is incorporated by reference in its entirety.

### FIELD OF THE INVENTION

The present invention relates to systems and methods using liquid titanium fabrics and, more specifically, to systems and methods for medical and therapeutic applications of liquid titanium fabrics.

### BACKGROUND OF THE INVENTION

The Phild process for liquid titanium implements has been around since the 1980's. The Phild process claims to incorporate small amounts of titanium into fabric. Products made by the Phild process are known in fields such as professional sports, as some major league baseball players use nylon-coated necklaces that incorporate this technology. The health benefits, if any, of these necklaces has not been rigorously tested.

Far infrared (FIR) is a region in the infrared spectrum of electromagnetic radiation. While definitions vary, far infrared is often defined as any radiation with a wavelength of approximately 15 μm to approximately 1 mm. Far infrared rays have long been suspected to have therapeutic benefits. Several studies have examined the use of infrared saunas in the treatment of chronic health problems, such as high blood pressure, congestive heart failure, and rheumatoid arthritis, and found some evidence of benefit. None of these studies, however, have provided conclusive evidence for the benefits of infrared saunas; and it is unclear if the benefits were due to the sauna itself or to infrared rays.

Needs exist for improved systems and methods for medical and therapeutic applications using liquid titanium fabrics.

### DETAILED DESCRIPTION OF THE PREFERRED EMBODIMENTS

Systems and methods are described for using various implements and procedures for medical and therapeutic applications using liquid titanium fabrics. In certain embodiments, the tools and procedures may be used in conjunction with treatment of humans and/or animals. The examples described herein relate to humans, horses, dogs, and cats for illustrative purposes only. Embodiments may be used for any member of the equidae family, canine family, feline family, camelids, small mammals, birds, farm animals, zoo animals, reptiles, amphibians, sheep, pigs, etc.

Embodiments of the present invention may utilize liquid titanium fabric to make one or more articles that are used in a treatment process.

The inventor has discovered that fabric materials that incorporate titanium have unexpectedly beneficial properties. While not wishing to be bound by any particular scientific theory, the inventor presently believes this is because the titanium reflects or radiates infrared radiation onto the skin.

Liquid titanium fabric may incorporate metallic titanium into one or more types of fabric. In certain embodiments, titanium particles may be combined with nano- or micro-sized TiO₂ under very high temperatures. In certain embodiments, the titanium particles may be magnetic. The nano- or micro-sized TiO₂ may be from various sources, including crystalline sources, such as rutile, anatase, brookite, ilmenite, etc. Exemplary temperatures for incorporation may be up to and/or above 1500° C. The resulting titanium particles may be bound into material fibers by a process using electric current to stabilize the product. The resulting titanium particles may be magnetic. Material fibers may include various types of fibers including natural, synthetic, and blended fibers. In certain embodiments, the material fibers may include polyester, nylon, cotton, wool, bamboo, etc. Fibers that may shrink may be preshrunk prior to binding with titanium particles. Inside the material fibers, the titanium particles may remain permanently bound and may not wash out with soap or water.

The liquid titanium fabric may comprise approximately 30% to approximately 100% liquid titanium fibers, more preferably approximately 38% to approximately 100% liquid titanium fibers, more preferably approximately 46% to approximately 100% liquid titanium fibers, more preferably approximately 57% to approximately 100% liquid titanium fibers, and more preferably approximately 63% to approximately 100% liquid titanium fibers. In certain embodiments, the liquid titanium fabric may include more than approximately 50% liquid titanium fibers. In certain embodiments, the liquid titanium fabric may include approximately 100% liquid titanium fibers. The percentage of liquid titanium fibers may be based on a total of all fiber types. Other types of fibers may be combined with the liquid titanium fibers. For example, a liquid titanium fabric may include approximately 60% liquid titanium polyester fibers, approximately 38% non-liquid titanium polyester fibers, and approximately 2% spandex.

In certain embodiments, the effects of the liquid titanium fabric may begin to appear at different speeds depending on the percentage of liquid titanium fibers in the fabric. For example, fabrics with percentages of liquid titanium fibers greater than approximately 50% may show effects faster than fabrics with lower percentages of liquid titanium fibers. Effects in fabrics with higher percentages of liquid titanium fibers may appear in approximately 5 minutes to approximately 10 minutes, but results can vary depending on the article, patient, and ailment. In certain embodiments, effects may begin to show at less than 5 minutes or greater than approximately 10 minutes, greater than approximately 20 minutes, greater than approximately 30 minutes, etc.

Fabrics may be made in a variety of weights. The weight of the fabric may vary depending on the amount of liquid titanium fibers and other factors.

The liquid titanium fabrics described herein may have various health benefits. While not wanting to be constrained by any theory, the bound titanium may produce or reflect far infrared rays.

The fabric or its use may result in warming the area of coverage. Far infrared rays may penetrate into the skin's subcutaneous layers, where it converts into radiant heat (infrared energy). This may raise body temperature. Increased body temperature may be localized or generalized over the user's body.

The fabric or its use may also improve blood circulation. Far infrared rays may cause water molecules to resonate, which may increase water hydration capacity in blood vessels. Well-hydrated blood vessels may expand and thin blood to improve circulation.

The fabric or its use may also detoxify. Warmth from radiant heat (infrared energy) may help flush out toxic substances, heavy metals, and other cell waste products through sweat and sebaceous (fat) glands in the human skin.

The fabric or its use may also improve immune response, increase metabolism, and aid in the production of nitric oxide. White blood cells may require nitric oxide in the defense against cancerous cells. Nitric oxide may be a critical component of the immune system and may allow cellular healing to take place.

The fabric or its use may also aid in cardiovascular conditioning. It is known that infrared stimulation is an ideal way to maintain cardiovascular conditioning. Cardiovascular conditioning may improve with use of the fabric.

The fabric or its use may also have a calming effect. Negative ions may be produced by the fabric. The negative ions may induce alpha brain waves, spreading from the occipital area to the parietal, temporal, and even reaching frontal lobes. Alpha waves, distributed evenly on right and left hemispheres of the brain, may create an overall calming effect.

The fabric or its use may increase awareness and sensitivity to external stimuli. Negative ions may promote alpha brain waves and/or increase brain wave amplitude.

The fabric or its use may improve nervous system regulation. Serotonin affects human neurovascular, endocrinal, and metabolic systems. This may in turn affect such things as sleep quality, depression, anxiety, headaches, etc. Negative ions may regulate serotonin levels.

The fabric or its use may also improve the health of diabetics without the use of compression. The fabric or its use may regulate blood sugar levels in patients, including patients that are diabetic or pre-diabetic.

The fabric or its use may be ultraviolet (UV) light blocking. UVA radiation may cause skin aging, wrinkles, DNA damage, and skin cancer. UVB radiation may cause skin cancer, DNA mutation, etc. The fabric may block both UVA and UVB radiation. The UV rating may be SPF 15 or higher, 30 or higher, 45 or higher, 50 or higher, 55 or higher, 75 or higher, 100 or higher, etc.

The fabric may have additional beneficial features, such as deodorization, antibacterial properties, antimicrobial properties, moisture wicking properties, etc.

In humans and other animals, the fabric may be provided as a variety of articles directed at specific treatments. For example, arthritis of the hand may be treated by forming the fabric into gloves to fit one or both hands. A medical brace may be provided for various body parts. The medical brace may have an insert, removable liner, or lining made of liquid titanium fabric; or the body of the brace may be made of liquid titanium fabric. The insert or lining may be removable and replaceable or washable to allow subsequent uses with the same or different patients.

In humans and other animals, ear plugs may be made at least partially of liquid titanium fabric. For example, a foam or other compressive ball may be covered with liquid titanium fabric. The ball may be compressed to fit into the user's ear. Friction between the liquid titanium fabric and the user's ear may prevent the ear plug from coming loose and/or falling out. Ear plugs for horses have been shown to reduce anxiety and have a calming effect.

An article may include one or more layers of liquid titanium fabric. Other materials, fabrics, etc., may be included in the article. For example, fastening mechanisms may be provided to attach and/or secure the article to a patient. These may include, but are not limited to, buttons, snaps, hook and loop closures, zippers, etc. Articles may be sized for a particular type of patient and/or size of patient. At least one of the one or more layers may interface with a patient, whether human or animal. One or more articles may be used together to improve treatment or treat different parts of a patient. The one or more articles may preferably be applied directly to a patient, such as against the skin. Alternatively, the fabric may be applied to hair or fur. Alternatively, the fabric may be applied with an intermediary between the fabric and the patient. For example, an intermediary layer of non-liquid titanium fabric may be located between the liquid titanium fabric and the patient. The layer of non-liquid titanium fabric may be an article of clothing. Preferably, the article may be applied to at least a portion of the region affected by the ailment or symptoms of the ailment. For example, a back brace may cover the region of the back affected by pain. Placing an article at a remote region may also provide reduction or elimination of symptoms of an ailment.

The fabric may be supplied with external energy to increase its effectiveness. For example, electrically resistant wires may be implanted in the fabric to permit an electric current to generate heat, which may increase the effectiveness of the article. Alternatively, the fabric may be placed in a heated environment such as an oven, dryer, or heater and then removed to wrap around or otherwise contact a patient or area of interest. Similarly, the fabric may be impregnated with a heat-causing chemical such as camphor to generate heat. Other methods of treating and/or heating the fabric may be used to increase effectiveness.

The article may be applied to a patient for a predetermined duration of time or as needed. The predetermined duration of time may be for the number of days and/or for a set time for each session. For example, the predetermined duration of time may be once a day for one hour, twice a day for thirty minutes each, three times per day for ten minutes each, etc. The article may be applied for one day, one week, one month, or as long as the ailment or symptoms of the ailment persist.

The article also can be applied as needed based on occurrence of symptoms. The article may also be applied preemptively to prevent or reduce future symptoms associated with an ailment.

The article may be one or more of the following: blankets, coolers for horses and other animals, bedding, cushions, gloves, wraps, medical braces, hats, headbands, bandannas, scarves, shirts, leggings, pants, shorts, underwear, socks, shoes, shoe inserts, shoe liners, slippers, hock boots, stifle wrap, leg wrap, knee wrap, ankle wrap, pastern wrap, saddle pad, neck cover, ear cover, ear plug, animal beds etc. The article may also include ear plugs, such as foam plugs of various shapes and sizes covered by liquid titanium fabric. Various forms of sports equipment and apparel may also be made of liquid titanium fabrics. Sport equipment may include, but is not limited to, jerseys, shirts, hats, helmet liners, bandanas, headbands, gloves, braces, brace liners, pads, pad liners, socks, wraps, undergarments, swimsuits, jackets, pants, shorts, etc.

In addition, one or more articles of horse equipment may be made out of titanium, or may include titanium components. For example, bridles and/or bits may include at least some titanium. In certain embodiments, one or more articles of horse equipment may be covered with liquid titanium fabric. In certain embodiments, these one or more articles of horse equipment may be in contact or in proximity to the horse during use.

Ailments may include stress, anxiety, headaches, migraines, cramping, inflammation, lack of energy, insomnia, diabetes, poor sleep quality, arthritis, healing of incisions, healing of wounds, healing of fractures, polysaccharide storage myopathy, body pain, laminitis, poor circulation, poor hair growth, stall walking, stall weaving, headshaking syndrome, bad shipping, wash out before competition, fear of shots, etc. Ailments may be acute and/or chronic.

The systems and methods described herein may also be useful for various non-medical treatments and/or therapeutic effects. For example, liquid titanium fabrics may improve alertness, focus, and/or concentration. Liquid titanium fabrics may reduce motion sickness.

The systems and methods described herein may also be useful in military applications. In embodiments, liquid titanium fabrics may be used to increase alertness, focus, and/or concentration in soldiers while on duty. In embodiments, liquid titanium fabrics may be used to treat military personnel, such as soldiers suffering from post-traumatic stress disorder (PTSD).

Experiments have been made using various articles described above with human patients. The following are exemplary results of use of articles for treatment of ailments in humans:
Reduction of stress and anxiety;
Reduction of migraine headaches;
Reduction of cramping in calves and feet;
Reduction of inflammation in legs and ankles;
Head coverings have produced an energy boost;
Bedding has produced a good night's sleep;
Gloves reduced arthritic pain in hands;
Healed incisions faster;
Reduced chemotherapy headaches;
Reduction of arthritic aches and pain in: neck, back, shoulder, knee, wrist, ankle, and hips;
Improved focus generally, and in particular improved concentration on work and computers, and while playing tennis, golf, and shooting;
Reduced appetite;
Reduced motion sickness; and
Improved alertness in general, and in particular when driving and when tired.

Experiments have been made using various articles described above with horse patients. The following are exemplary results of use of articles for treatment of ailments in horses:
Leg wraps reduced inflammation;
Knee boots reduced arthritic pain and inflammation;
Hock boots reduced arthritic pain and inflammation;
Stifle wrap reduced arthritic pain and inflammation;
Improved blood flow to feet to reduce laminitis issues;
Pastern wrap reduced inflammation;
Saddle pad reduced back pain;
Blanket reduced body pain and inflammation;
Neck cover reduced arthritic pain and inflammation;
Bed sheets improved circulation in neo-natal foals;
Ear covers reduced anxiety;
Improved hair growth;
Improved immune system with less snots;
Improved polysaccharide storage myopathy (PSSM);
Blind horses adapted better to surroundings;
Horses focused on their job better;
Reduced anxiety;
Less gastric medication used;
Stall walkers and weavers have stopped;
Headshaking syndrome has improved;
Bad shippers relaxed;
Reduced wash out before competition;
Reduced fear of shots;
Stallions behaved more mannerly; and
Fractures healed more quickly.

Experiments have been made using various articles described above with dog and/or cat patients. The following are exemplary results of use of articles for treatment of ailments in dogs and/or cats:
Reduced arthritic pain in joints;
Reduced healing time for open wounds;
Reduced healing time for incisions;
Reduced anxiety;
Cat pads relaxed hyper cats;
Reduced requirement for arthritic medications;
Reduced barking; and
Improved quality and quantity of sleep.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

What is claimed is:

1. A method of reducing symptoms of an ailment, the method comprising:
providing an article comprising liquid titanium fabric; and
applying the fabric to a patient for a period of time effective to reduce symptoms of an ailment.

2. The method of claim 1, wherein the article is selected from the group consisting of: blankets, coolers, bedding, cushions, gloves, wraps, medical braces, hats, headbands, bandannas, scarves, shirts, leggings, pants, shorts, underwear, socks, shoes, shoe inserts, shoe liners, slippers, hock boots, stifle wrap, leg wrap, knee wrap, ankle wrap, pastern wrap, saddle pad, neck cover, ear cover, ear plugs, animal beds, and combinations thereof.

3. The method of claim 2, wherein the article is one or more gloves.

4. The method of claim 1, wherein the fabric comprises between approximately 60%-100% titanium fibers.

5. The method of claim 1, wherein the patient is a human.

6. The method of claim 1, wherein the patient is a horse.

7. The method of claim 1, wherein the patient is a dog.

8. The method of claim 1, wherein the ailment is selected from the group consisting of stress, anxiety, headaches, migraines, cramping, inflammation, lack of energy, insomnia, diabetes, poor sleep quality, arthritis, healing of incisions, healing of wounds, healing of fractures, polysaccharide storage myopathy, body pain, laminitis, poor circulation, poor hair growth, stall walking, stall weaving, headshaking syndrome, bad shipping, wash out before competition, fear of shots, and combinations thereof.

9. The method of claim 8, wherein the ailment is arthritis.

10. The method of claim 1, wherein the article is a glove and the ailment is arthritis.

11. The method of claim 1, wherein the fabric is applied directly to the patient.

12. The method of claim 1, wherein the fabric is applied to at least one intermediary material between the patient and the fabric.

13. The method of claim 12, wherein the intermediary material is a layer of clothing.

14. The method of claim 1, wherein the fabric is antimicrobial.

15. The method of claim 1, wherein the fabric has a UV rating of at least approximately SPF 15.

16. The method of claim 1, wherein the fabric is deodorizing.

17. An apparatus comprising:
one or more layers of liquid titanium fabric; and
an interface with a patient, wherein the patient has at least one ailment.

18. The apparatus of claim 1, wherein the apparatus is a glove for a human.

19. The apparatus of claim 1, wherein the apparatus is an ear cover or ear plug for a horse.

20. The apparatus of claim 1, wherein the apparatus is a medical brace.
